(19) United States
(12) Patent Application Publication
(10) Pub. No.: US 2023/0067182 A1
(43) Pub. Date: Mar. 2, 2023

(54) Data Processing Device and Method, and Computer Readable Storage Medium

(71) Applicant: BOE TECHNOLOGY GROUP CO., LTD., Chaoyang District, Beijing (CN)

(72) Inventors: Zhihao Chen, Beijing (CN); Dong Chai, Beijing (CN); Haohan Wu, Beijing (CN); Hong Wang, Beijing (CN)

(21) Appl. No.: 17/252,326

(22) PCT Filed: Nov. 29, 2019
(86) PCT No.: PCT/CN2019/121869

§ 371 Date: Dec. 15, 2020

(51) Int. Cl. G06F 16/27; G06F 16/23; G06F 16/25

(52) U.S. Cl. CPC G06F 16/27; G06F 16/2365; G06F 16/254

(57) ABSTRACT

Provided is a data processing device and method, and a computer-readable storage medium. The data processing device includes a memory and a processor performing following operations based on instructions stored in the memory: extracting first data from a first data table in a relational factory database at a first extraction cycle a duration of which is greater than 1 minute, the first data including data updated by the factory during the first extraction cycle; storing the first data into a second data table of a distributed storage system to form second data; inserting the second data into a third data table of the distributed storage system to form third data after data integrating the second data; and calling data in the third data table for data analysis processing at a first analysis cycle a duration of which is not smaller than the duration of the first extraction cycle.

### CROSS-REFERENCE TO RELATED APPLICATIONS

The present application is a U.S. National Stage Application under 35 U.S.C. § 371 of International Patent Application No. PCT/CN2019/121896, filed on Nov. 29, 2019, the disclosure of which is incorporated by reference herein in its entirety.

### TECHNICAL FIELD

The present disclosure relates to a data processing device and method, and a computer-readable storage medium.

### BACKGROUND

In the field of big data processing, required data needs to be extracted from a relational database into a big data platform. The big data platform processes the extracted data according to service logic to meet the data requirement of upper layer application.

In the related art, required data is extracted from a relational database into a large data platform by using an Extract-Transform-Load (ETL) tool.

### SUMMARY

According to a first aspect of embodiments of the present disclosure, provided is a data processing device comprising: at least one memory configured to store instructions; and at least one processor coupled to the at least one memory, and configured to, based on the instructions, perform following steps: extracting first data from a first data table in a relational factory database at a first extraction cycle, wherein the first data comprises data updated by the factory during the first extraction cycle, and a duration of the first extraction cycle is greater than 1 minute, storing the first data into a second data table of a distributed storage system to form second data, inserting the second data into a third data table of the distributed storage system to form third data after performing data integration on the second data, and calling data in the third data table for data analysis processing at a first analysis cycle, wherein a duration of the first analysis cycle is not smaller than the duration of the first extraction cycle.

In some embodiments, after the second data is inserted into the third data table to form the third data, further comprising: checking, during a preset time period, the data inserted into the third data table during a first processing cycle with the data stored into the second data table during the first processing cycle, such that the data inserted into the third data table during the first processing cycle is consistent with the data updated in the first data table during the first processing cycle, wherein the duration of the first analysis cycle is greater than a preset threshold during the preset time period.

In some embodiments, the duration of the first extraction cycle ranges from 10 minutes to 1 day.

In some embodiments, checking the data inserted into the third data table during the first processing cycle with the data stored into the second data table during the first processing cycle comprises: performing at least one of deduplication or missing data supplement on the data inserted into the third data table during the first processing cycle with the data stored into the second data table during the first processing cycle.

In some embodiments, the first data table comprises a first data sub-table and a second data sub-table, and the second data table comprises a third data sub-table and a fourth data sub-table, the first data sub-table comprising first sub-data in the factory database after modification, the second data sub-table comprising second sub-data that is removed during the modification; extracting the first data from the first data table at the first extraction cycle comprises: extracting the first sub-data from the first data sub-table, and extracting the second sub-data from the second data sub-table at the first extraction cycle; storing the first data into the second data table comprises: storing the first sub-data into the third data sub-table to form third sub-data, and storing the second sub-data into the fourth data sub-table to form fourth sub-data; and inserting the second data into the third data table after performing data integration on the second data comprises: inserting the third sub-data into the third data table after performing data integration on the third sub-data.

In some embodiments, checking the data inserted into the third data table with the data stored in the second data table comprises: filtering the data inserted into the third data table during a second processing cycle with the data stored into the fourth data sub-table in the second processing period to remove the fourth sub-data inserted into the third data table during the second processing cycle, wherein a duration of the second processing cycle is greater than the duration of the first processing cycle.

In some embodiments, the second data comprises fifth sub-data and sixth sub-data with a compression format; and inserting the second data into the third data table after performing data integration on the second data comprises: performing format conversion on the sixth sub-data to obtain seventh sub-data with a preset data format, associating the fifth sub-data and the seventh sub-data according to a data identifier to obtain fourth data, and inserting the fourth data into the third data table after performing data integration on the fourth data.

In some embodiments, performing format conversion on the sixth sub-data comprises: extracting the sixth sub-data from the second data; and sending the sixth sub-data to a Linux server such that the Linux server performs format conversion on the sixth sub-data to obtain the seventh sub-data with the preset data format.

In some embodiments, the compression format is a BLOB format.

According to a second aspect of embodiments of the present disclosure, provided is a data processing method, comprising: extracting first data from a first data table in a relational factory database at a first extraction cycle, wherein the first data comprises data updated by the factory during the first extraction cycle, and a duration of the first extraction cycle is greater than 1 minute; storing the first data into a second data table of a distributed storage system to form second data; inserting the second data into a third data table of the distributed storage system to form third data after performing data integration on the second data; and calling data in the third data table for data analysis processing at a first analysis cycle, wherein a duration of the first analysis cycle is not smaller than the duration of the first extraction cycle.

In some embodiments, after inserting the second data into a third data table of the distributed storage system to form third data after performing data integration on the second data, the data processing method further comprises: checking, during a preset time period, the data inserted into the third data table during a first processing cycle with the data stored into the second data table during the first processing cycle, such that the data inserted into the third data table during the first processing cycle is consistent with the data updated in the first data table during the first processing cycle, wherein a duration of the first analysis cycle is greater than a preset threshold during the preset time period.

In some embodiments, the duration of the first extraction cycle ranges from 10 minutes to 1 day.

In some embodiments, the first data table comprises a first data sub-table and a second data sub-table, and the second data table comprises a third data sub-table and a fourth data sub-table, the first data sub-table comprising first sub-data in the factory database after modification, the second data sub-table comprising second sub-data that is removed during the modification; extracting the first data from the first data table at the first extraction cycle comprises: extracting the first sub-data from the first data sub-table, and extracting the second sub-data from the second data sub-table at the first extraction cycle; storing the first data into the second data table comprises: storing the first sub-data into the third data sub-table to form third sub-data, and storing the second sub-data into the fourth data sub-table to form fourth sub-data; inserting the second data into the third data table after performing data integration on the second data comprises: inserting the third sub-data into the third data table after performing data integration on the third sub-data; and checking the data inserted into the third data table with the data stored into the second data table comprises: filtering the data inserted into the third data table during a second processing cycle with the data stored into the fourth data sub-table in the second processing period to remove the fourth sub-data inserted into the third data table during the second processing cycle, a duration of the second processing cycle is greater than the duration of the first processing cycle.

In some embodiments, the second data comprises fifth sub-data and sixth sub-data with a compression format; and inserting the second data into the third data table after performing data integration on the second data comprises: extracting the sixth sub-data from the second data, sending the sixth sub-data to a Linux server such that the Linux server performs format conversion on the sixth sub-data to obtain a seventh sub-data with the preset data format, associating the fifth sub-data and the seventh sub-data according to a data identifier to obtain fourth data, and inserting the fourth data into the third data table after performing data integration on the fourth data.

According to a third aspect of embodiments of the present disclosure, provided is a computer readable storage medium storing computer instructions which, when executed by a processor, perform the data processing method according to any one of the above embodiments.

Other features of the present disclosure and advantages thereof will become apparent from the following detailed description of exemplary embodiments thereof, which proceeds with reference to the accompanying drawings.

### BRIEF DESCRIPTION OF THE DRAWINGS

The accompanying drawings, which constitute part of this specification, illustrate exemplary embodiments of the present disclosure and, together with this specification, serve to explain the principles of the present disclosure.

The present disclosure can be understood more clearly from the following detailed description with reference to the accompanying drawings, in which:

FIG. 1 is a schematic diagram showing a data processing scenario according to an embodiment of the present disclosure;

FIG. 2 is a schematic flow chart showing a data processing method according to an embodiment of the present disclosure;

FIG. 3 is a schematic flow chart showing a data processing method according to another embodiment of the present disclosure;

FIG. 4 is a schematic diagram showing an architectural of the embodiment shown in FIG. 3;

FIG. 5 is a schematic flow chart showing a data processing method according to still another embodiment of the present disclosure;

FIG. 6 is a schematic diagram showing an architectural of the embodiment shown in FIG. 5;

FIG. 7 is a schematic flow chart showing a data processing method according to yet still another embodiment of the present disclosure;

FIG. 8 is a schematic diagram showing an architectural of the embodiment of FIG. 7;

FIG. 9 is a schematic structural diagram showing a data processing device according to an embodiment of the present disclosure.

It should be understood that the dimensions of the various parts shown in the accompanying drawings are not necessarily drawn according to the actual scale. In addition, the same or similar reference signs are used to denote the same or similar components.

### DETAILED DESCRIPTION

Various exemplary embodiments of the present disclosure will now be described in detail with reference to the accompanying drawings. The following description of the exemplary embodiments is merely illustrative and is in no way intended as a limitation to the present disclosure, its application or use. The present disclosure may be implemented in many different forms, which are not limited to the embodiments described herein. These embodiments are provided to make the present disclosure thorough and complete, and fully convey the scope of the present disclosure to those skilled in the art. It should be noticed that: relative arrangement of components and steps, material composition, numerical expressions, and numerical values set forth in these embodiments, unless specifically stated otherwise, should be explained as merely illustrative, and not as a limitation.

The use of the terms “first”, “second” and similar words in the present disclosure do not denote any order, quantity or importance, but are merely used to distinguish between different parts. A word such as “comprise”, “have” or variants thereof means that the element before the word covers the element(s) listed after the word without excluding the possibility of also covering other elements.

Unless otherwise defined, all terms (including technical and scientific terms) used herein have the same meanings as the meanings commonly understood by one of ordinary skill in the art to which the present disclosure belongs. It should also be understood that terms as defined in general dictionaries, unless explicitly defined herein, should be interpreted as having meanings that are consistent with their meanings in the context of the relevant art, and not to be interpreted in an idealized or extremely formalized sense.

Techniques, methods, and apparatus known to those of ordinary skill in the relevant art may not be discussed in detail, but where appropriate, these techniques, methods, and apparatuses should be considered as part of this specification.

The inventors have found through research that different big data analysis applications in manufacturing industry have different requirements on real-time performance of data. The quasi-real-time requirement of data delay on the order of milliseconds can be realized by technologies such as flink, kafka, or spark timing. Batch data processing without time-efficiency requirements can be implemented by Hive components. In manufacturing industry, however, the time interval required by the applications may be on the order of minutes, such as 5 minutes, 30 minutes, or one hour. For example, whether the production capacity of each device on the production line conforms to the production schedule is analyzed every half hour, or the product yield condition and the cause of failure in one hour are analyzed based on the production data in this one hour. At present, however, Hive cannot be used to meet the requirement of application synchronization on the order of minutes.

Accordingly, the present disclosure proposes a solution to meet the minute-level synchronization requirements of users on data in the process of extracting data from a relational database to a distributed storage system.

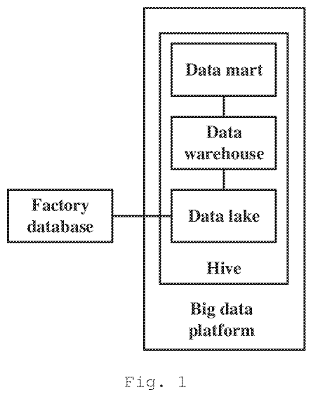

FIG. 1 is a schematic diagram showing a data processing scenario according to an embodiment of the present disclosure.

As shown in FIG. 1, a big data platform based on a Distributed File System (e.g., Hadoop Distributed File System, HDFS for short) is present in a data processing scenario. Big data technology based on the distributed file system uses a software framework of distributed processing and allows the use of a plurality of cheap hardware devices to construct a large cluster to process mass data. Hive is a data warehouse tool based on Hadoop, which is used for data extraction, transformation and loading (ETL). Hive defines a simple SQL-like query language, allowing users familiar with SQL to query data, and also allowing developers familiar with MapReduce to develop customized mappers and reducers to process complex analysis work which cannot be completed by built-in mappers and reducers. Hive has no special data storage format and indexes for data. The users can freely organize tables in Hive to process data in a database.

For a factory, a lot of data is generated during the production process of the factory. These data are stored in the factory database. The factory database is mostly a relational database. The database mainly adopts a grid computing technology of a Relational Database Management System (RDBMS). Through the RDBMS grid computing, a problem which can only be solved with huge computer capacity is divided into a plurality of small parts, then the plurality of small parts are distributed to a plurality of computers for processing, and finally the computing results are integrated to obtain a final result. For example, in an Oracle RAC (Real Application Cluster), all servers can directly access all data in the database. However, the hardware expansion space of the application system based on RDBMS grid computing is limited. Thus, when the data volume reaches a certain order of magnitude, the efficiency of processing massive data is very low due to the bottleneck of input/output of the hard disk. The parallel processing of the distributed file system can meet the requirement of ever-increasing data storage and processing. Therefore, when massive data of a factory is analyzed, data of a factory database needs to be extracted into the distributed file system.

As shown in FIG. 1, data is first extracted from the factory database into the data lake of the Hive data warehouse of the big data platform. This step may be understood as extracting data from a first data table in the factory database into a second data table of Hive. The data in the second data table is the same as the data in the first data table except that the storage format and the storage location of the data are changed. And then, processes such as layering process, integration process, or the like are performed on the data in the data lake according to corresponding service logic, and the processing result is stored into the data warehouse to meet the requirement of upper-layer application on the data. This step may be understood as storing the data in the second data table into the third data table after the data is performed data integration. The data integration comprises data addition and deletion, multi-table association and the like. The third data table is simpler and more accurate than the second data table. For example, based on the requirement of product failure cause analysis, the third data table after data integration comprises related factors such as dimension columns (time, factory, equipment, operator, and the like), attribute columns (factory location, equipment service life, number of defect dots, abnormal parameters, energy consumption parameters, process duration, and the like) involved in the factory automation process. And then, preprocessing such as dimension table design, abnormal value processing, discretization processing, and normalization processing is performed on the data in the data warehouse by using a data preprocessing platform. Further, the extracted data is subjected to feature analysis by using a correlation algorithm. For example, data feature extraction is performed by using methods such as Pearson Correlation Coefficient, One-Hot Encoding, or the like to find features meeting service requirements and implement services such as query and analysis. In addition, algorithms such as random forest, XGBOOST or the like are used for modeling and parameter tuning so as to obtain prediction information or specific conclusions required by service requirements and realize services such as speculation and inference.

In response to a query requirement input by a user, a data virtualization platform such as TIBCO DV is called to carry out task planning on the requirement, to determine to directly call data in the Hive data table of the big data platform, or call a corresponding algorithm model to process related data to obtain a result meeting the requirement of the user. And finally, the corresponding result is presented to the user.

The following describes solutions for extracting data from a relational database to a distributed storage system in detail with reference to specific embodiments.

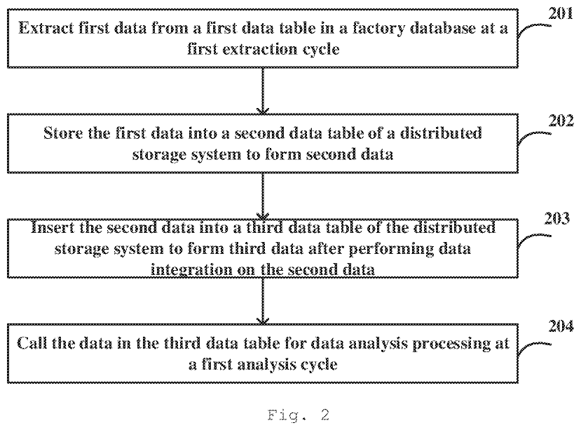

FIG. 2 is a schematic flow chart showing a data processing method according to an embodiment of the present disclosure. The data processing method is performed by a data processing device. The data processing device comprises at least one memory and at least one processor. The memory is configured to store instructions. The processor is coupled with the at least one memory. The processor is configured to perform the following operations based on the instructions stored in the memory.

At step 201, first data in a first data table in a factory database is extracted at a first extraction cycle.

The first data comprises data updated by the factory during in first extraction cycle. Operations such as adding, deleting, modifying, or the like is performed by the factory to update data. The factory database is a relational database. The duration of the first extraction cycle is greater than 1 minute.

In some embodiments, the factory database is an Oracle database and the first data table is an Oracle data table.

In some embodiments, the duration of the first extraction period is determined according to actual service requirements, and may also be periodically changed according to different time period requirements of the same service.

In some embodiments, the duration of the first extraction cycle ranges from 10 minutes to 1 day.

For example, when material accumulation condition of each process station in a production process is analyzed and predicted, production data can be extracted every half hour to 1 hour for the upper layer application to perform corresponding prediction analysis, thereby preventing the material accumulation. For another example, to analyze production schedule, the production data may be extracted every half or 1 day to obtain the production data and the data of the production schedule for the upper layer application to analyze the production schedule. For still another example, to analyze production defects or failures in a production line, the duration of the first extraction cycle may be reduced, such as extracting production data every 10 minutes, to perform failure prediction analysis and reduce production accidents.

It should be noted that, in the first data table, each data has a corresponding time stamp. Thereby, by using the time stamp, the incremental data in the first data table can be extracted. Data extraction can be realized by using data extraction tools such as Sqoop, Datax, Kettle and the like.

At step 202, the first data is stored into a second data table of a distributed storage system to form second data.

At step 203, the second data is inserted into a third data table of the distributed storage system to form third data after being performed data integration.

In some embodiments, the second data table and the third data table are Hive data tables. For example, the second data table is located in the data lake of FIG. 1 and the third data table is located in the data warehouse or data mart of FIG. 1.

Here, it should be noted that the second data is inserted into a third data table means that operations such as overwriting or deleting of data in the third data table are not performed in the process of writing the second data into the third data table, to ensure that an application program does not generate an error when processing the data due to overwriting or deleting of the data.

At step 204, data in the third data table is called for data analysis processing at a first analysis cycle. The first analysis cycle is not shorter than the first extraction cycle.

It should be noted that, since the data in the third data table is updated at the first extraction cycle, when the first analysis cycle is not shorter than the first extraction cycle, the situation of repeating the analysis of the same data will not occur.

In some embodiments, after the second data is inserted into the third data table of the distributed storage system to form the third data, data inserted into the third data table during a first processing cycle is checked, in a preset time period, with data stored into the second data table during the first processing cycle, such that the data inserted into the third data table in the first processing period is consistent with data updated in the first data table in the first processing period.

It should be noted that, in the process of inserting the second data into the third data table, due to instability of the distributed storage system itself, abnormality may occur in the insertion operation, which leads to data abnormality in the third data table. The data in the third data table is checked by using the data in the second data table to keep the data in the third data table consistent with the data in the first data table.

In some embodiments, the duration of the first processing cycle is greater than the duration of the first extraction cycle. For example, the duration of the first extraction cycle is 30 minutes and the duration of the first processing cycle is 3 days.

In some embodiments, the duration of the first processing cycle is greater than the interval of preset time periods and the data update frequency period, such that the data inserted into the third data table during the first processing cycle is consistent with the data updated in the first data table during the first processing cycle.

In some embodiments, in the preset period time, the duration of the first analysis cycle is greater than a preset threshold.

It should be noted that, in order to avoid the data analysis from being affected by the check on the data in the third data table, a time period with a low frequency of data analysis is selected as the preset time period. For example, the frequency of analyzing the data in the third data table is low every night, so the preset time period may be set as time period at night (for example, between 3 and 4 o'clock in the morning).

In some embodiments, in the case where the data stored into the second data table during the first processing cycle is not consistent with the data inserted into the third data table during the first processing cycle, the data inserted into the third data table during the first processing cycle is overwritten with the data stored into the second data table during the first processing cycle to perform at least one of deduplication or missing data supplement on the data inserted into the third data table.

The data extracted from the first data table mainly comprises three types of data. The first type of data does not change after being stored into the relational database. The second type of data will change after being stored into the relational database. The third type of data is data comprising a preset format. The processing schemes of the three types of data will be explained below, respectively.

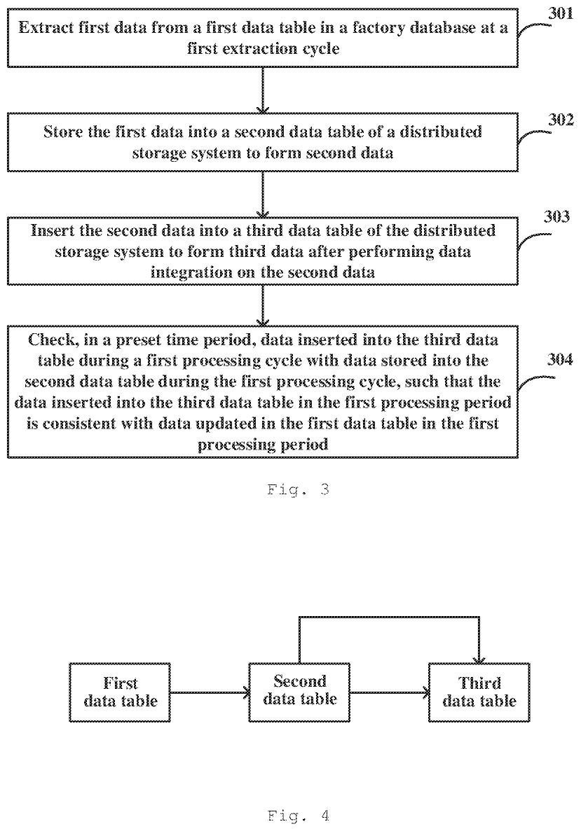

FIG. 3 is a schematic flow chart showing a data processing method according to another embodiment of the present disclosure. The data processing method is performed by a processor based on instructions stored in a memory.

At step 301, first data in a first data table in a factory database is extracted at a first extraction cycle.

The first data comprises data updated by the factory during in first extraction cycle. Operations such as add operation, delete operation, modify operation, or the like is performed by the factory to update data. The factory database is a relational database. The duration of the first extraction cycle is greater than 1 minute.

Here, it should be noted that the data extracted from the first data table is the first type of data, that is, data that will not change after being stored into the relational database.

In some embodiments, the factory database is an Oracle database and the first data table is an Oracle data table.

In some embodiments, the duration of the first extraction period is determined according to actual service requirements, and may also be periodically changed according to different time period requirements of the same service.

In some embodiments, the duration of the first extraction cycle ranges from 10 minutes to 1 day.

It should be noted that, in the first data table, each data has a corresponding time stamp. Thereby, by using the time stamp, the incremental data in the first data table can be extracted. Data extraction can be realized by using data extraction tools such as Sqoop, Datax, Kettle and the like.

At step 302, the first data is stored into a second data table of a distributed storage system to form second data.

At step 303, the second data is inserted into a third data table of the distributed storage system to form third data after being performed data integration.

In some embodiments, the second data table and the third data table are Hive data tables. For example, the second data table is located in the data lake of FIG. 1 and the third data table is located in the data warehouse or data mart of FIG. 1.

At step 304, data inserted into the third data table during a first processing cycle is checked, in a preset time period, with data stored into the second data table during the first processing cycle, such that the data inserted into the third data table in the first processing period is consistent with data updated in the first data table in the first processing period.

In some embodiments, the duration of the first processing cycle is greater than the duration of the first extraction cycle. For example, the duration of the first extraction cycle is 30 minutes and the duration of the first processing cycle is 3 days.

In some embodiments, the duration of the first processing cycle is greater than the interval of preset time periods and the data update frequency period, such that the data inserted into the third data table during the first processing cycle is consistent with the data updated in the first data table during the first processing cycle.

It should be noted that, in order to avoid the data analysis from being affected by the check on the data in the third data table, a time period with a low frequency of data analysis is selected as the preset time period. For example, the frequency of analyzing the data in the third data table is low every night, so the preset time period may be set as time period at night (for example, between 3 and 4 o'clock in the morning).

In some embodiments, in the case where the data stored into the second data table during the first processing cycle is not consistent with the data inserted into the third data table during the first processing cycle, the data inserted into the third data table during the first processing cycle is overwritten with the data stored into the second data table during the first processing cycle to perform at least one of deduplication or missing data supplement on the data inserted into the third data table.

In some embodiments, data in the third data table is called for data analysis processing at a first analysis cycle. The duration of the first analysis cycle is not smaller than the duration of the first extraction cycle.

It should be noted that, since the data in the third data table is updated at the first extraction cycle, when the duration of the first analysis cycle is not smaller than the duration of the first extraction cycle, the situation of repeating the analysis of the same data will not occur.

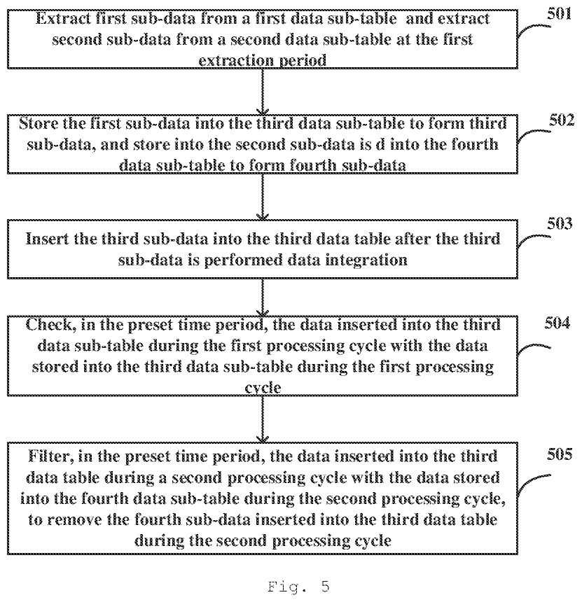

FIG. 4 is a schematic diagram showing an architectural of the embodiment shown in FIG. 3.

As shown in FIG. 4, first data is extracted from the first data table of the factory database every half hour. The first data is stored into the second data table of the distributed storage system to form second data. The second data is inserted into the third data table of the distributed storage system to form third data. Next, in a time period in which the third data table is used infrequently (for example, 3 to 4 am every day), the data inserted into the third data table in the last three days is checked with the second data table such that the data of the third data table is consistent with the data of the first data table in the last three days.

FIG. 5 is a schematic flow chart showing a data processing method according to still another embodiment of the present disclosure. The data processing method is performed by a processor based on instructions stored in a memory.

Compared to the embodiment shown in FIG. 3, in the embodiment shown in FIG. 5, the first data table comprises a first data sub-table and a second data sub-table. The second data table comprises a third data sub-table and a fourth data sub-table. The first data sub-table comprises modified first sub-data in the factory database after modification, and the second data sub-table comprises second sub-data removed during the modification. For example, the first data sub-table and second data sub-table are Oracle data tables. The third data sub-table and the fourth data sub-table are Hive data tables.

It should be noted that the data stored in the factory database may randomly update. In order to facilitate management of data, real-time data generated in industrial production is placed in the first data sub-table in the factory database, and data in the first data sub-table is updated. Old data deleted during the update process is placed in the second data sub-table in the factory database. That is, the data extracted from the first data table is the second type of data, that is, data that will change after being stored in the factory database.

For example, in the case where a defective product is produced on a production line, the product needs to be manufactured repeatedly. However, the number of the production history data corresponding to the product in the database will not increase, but the values of the production history data will be updated on the basis of the original values of the production history data. The time interval between repeated processes is uncertain and may be one day or one week. In this case, the data stored in the factory database will be randomly updated.

At step 501, first sub-data is extracted from a first data sub-table at a first extraction period and second sub-data is extracted from a second data sub-table at the first extraction period.

In some embodiments, the duration of the first extraction period is determined according to actual service requirements, and may also be periodically changed according to different time period requirements of the same service.

In some embodiments, the duration of the first extraction period ranges from 10 minutes to 1 day.

It should be noted that, in the first data sub-table and the second data sub-table, each data has a corresponding time stamp. By using the time stamp, the incremental data in the first data sub-table and the second data sub-table can be extracted. Data extraction can be realized by using data extraction tools such as Sqoop, Datax, Kettle and the like.

At step 502, the first sub-data is stored into the third data sub-table to form third sub-data, and the second sub-data is stored into the fourth data sub-table to form fourth sub-data.

At step 503, the third sub-data is inserted into the third data table after being performed data integration.

In some embodiments, the third data table is a Hive data table. For example, the third data sub-table and the fourth data sub-table are located in the data lake of FIG. 1, and the third data table is located in the data warehouse or the data mart of FIG. 1.

At step 504, in the preset time period, the data inserted into the third data sub-table during the first processing cycle is checked with the data stored into the third data sub-table during the first processing cycle.

For example, in the case where the data stored into the third data sub-table during the first processing cycle is not consistent with the data inserted into the third data table during the first processing cycle, the data inserted into the third data table during the first processing cycle is overwritten with the data stored into the third data sub-table during the first processing cycle to perform at least one of deduplication or missing data supplement on the data inserted into the third data table.

It should be noted that, in order to avoid the data analysis from being affected by the check on the data in the third data table, a time period with a low frequency of data analysis is selected as the preset time period. For example, the frequency of analyzing the data in the third data table is low every night, so the preset time period may be set as time period at night (for example, between 3 and 4 o'clock in the morning).

At step 505, in the preset time period, the data inserted into the third data table during a second processing cycle is filtered with the data stored into the fourth data sub-table during the second processing cycle, to remove the fourth sub-data inserted into the third data table during the second processing cycle.

It should be noted that, the duration of the second processing cycle is greater than the duration of the first processing cycle and the longest time interval for product repair. Since the data amount in the fourth data sub-table is small, the processing load can be reduced by filtering the data in the third data table with a longer processing cycle. For example, the duration of the first processing cycle is 3 days, and the duration of the second processing cycle is 30 days.

In some embodiments, the data in the third data table is called for data analysis processing at the first analysis cycle. The duration of the first analysis cycle is not smaller than the duration of the first extraction cycle.

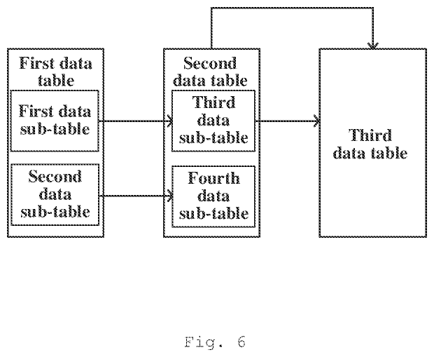

FIG. 6 is a schematic diagram showing an architectural of the embodiment shown in FIG. 5.

As shown in FIG. 6, the first sub-data is extracted from the first sub-table of data in the factory database every half hour, and second sub-data is extracted from the second sub-table of data in the factory database. The first sub-data is stored into the third data sub-table of the distributed storage system, and the second sub-data is stored into the fourth data sub-table of the distributed storage system. The third sub-data is inserted into the third data table of the distributed storage system. Next, in a time period in which the third data table is used infrequently (for example, 3 o'clock to 4 o'clock in the morning each day), the data inserted into the third data table in the last 3 days is subjected to missing data supplement processing and deduplication processing with the third data sub-table, and the data inserted into the third data table in the last 30 days is subjected to filtering processing with the fourth data sub-table, such that the data in the third data table is consistent with the data in the first data sub-table.

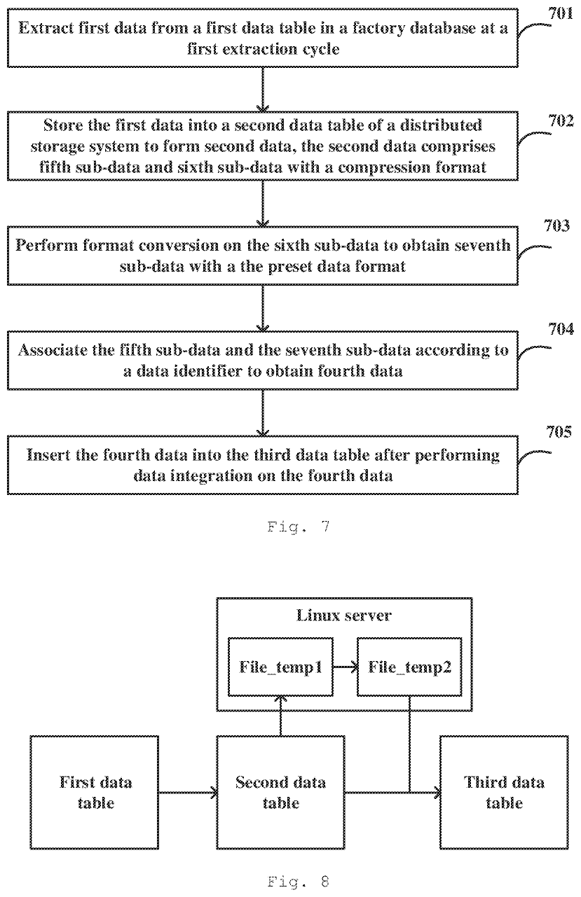

FIG. 7 is a schematic flow chart showing a data processing method according to yet still another embodiment of the present disclosure. The data processing method is performed by a processor based on instructions stored in a memory.

At step 701, first data in a first data table in a factory database is extracted at a first extraction cycle.

The first data comprises data updated by the factory during in first extraction cycle. Operations such as add operation, delete operation, modify operation, or the like is performed by the factory to update data. The factory database is a relational database. The duration of the first extraction cycle is greater than 1 minute.

It should be noted that the data extracted from the first data table is the third type of data, that is, the data comprises content with a preset data format.

In some embodiments, the factory database is an Oracle database and the first data table is an Oracle data table.

In some embodiments, the duration of the first extraction period is determined according to actual service requirements, and may also be periodically changed according to different time period requirements of the same service.

In some embodiments, the duration of the first extraction cycle ranges from 10 minutes to 1 day.

It should be noted that, in the first data table, each data has a corresponding time stamp. Thereby, by using the time stamp, the incremental data in the first data table can be extracted. Data extraction can be realized by using data extraction tools such as Sqoop, Datax, Kettle and the like.

At step 702, the first data is stored into a second data table of the distributed storage system to form second data. The second data comprises fifth sub-data and sixth sub-data. The fifth sub-data is of a preset data format, that is, a data format which can be normally presented in the distributed storage system. The sixth sub-data is of a compression format. For example, the compression format of the sixth sub-data is a BLOB format.

In some embodiments, the second data table is a Hive data table. For example, the second data table is located in the data lake of FIG. 1.

At step 703, format conversion is performed on the sixth sub-data to obtain seventh sub-data with the preset data format.

In some embodiments, the sixth sub-data is extracted from the second data. And the sixth sub-data is sent to a Linux server so that the Linux server can perform format conversion on the sixth sub-data to obtain the seventh sub-data with the preset data format.

At step 704, the fifth sub-data and the seventh sub-data are associated according to a data identifier to obtain fourth data.

At step 705, the fourth data is inserted into the third data table after being performed data integration.

In some embodiments, the third data table is a Hive data table. For example, the third data table is located in the data warehouse or the data mart of FIG. 1.

In some embodiments, the data in the third data table is called for data analysis processing at the first analysis cycle. The duration of the first analysis cycle is not smaller than the duration of the first extraction cycle.

For example, an abstract structure of the first data table in the factory database is shown in Table 1.

TABLE 1

| ID | BLOB_column | column |
|---|---|---|
| 123 | < blob > | AA |
| 456 | < blob > | BB |

The BLOB field comprises a plurality of parameters, and each of the plurality of parameters corresponding to a value. The corresponding abstract diagram of the design is shown in TABLE 2.

TABLE 2

| ID | Parameter(s) | Value |
|---|---|---|
| 123 | a | 1.1 |
| 123 | b | 3.5 |
| 456 | a | 2.9 |
| 456 | b | 1.8 |

The fifth sub-data (shown in TABLE 1) and the seventh sub-data (shown in TABLE 2) are associated according to the ID, and the obtained fourth data is inserted into the third data table. An abstract diagram of the design of the third data table is shown in TABLE 3.

TABLE 3

| ID | Parameter(s) | Value | column |
|---|---|---|---|
| 123 | a | 1.1 | AA |
| 123 | b | 3.5 | AA |
| 456 | a | 2.9 | BB |
| 456 | b | 1.8 | BB |

Thus, the fourth data comprises all the information in the first data.

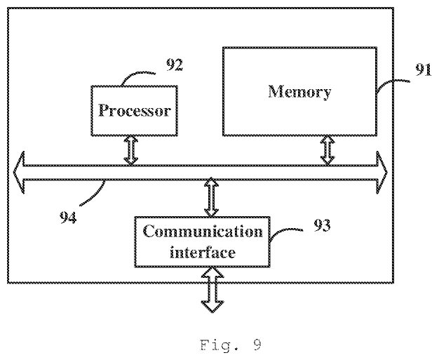

FIG. 8 is a schematic diagram showing an architectural of the embodiment of FIG. 7.

As shown in FIG. 8, first data is extracted from the first data table of the factory database every half hour. The first data is stored into the second data table of the distributed storage system to form second data. The second data comprises fifth sub-data with a preset data format and sixth sub-data with a BLOB format. The sixth data is downloaded to the Linux server and stored as a file_temp 1. The sixth data is processed by using a corresponding Java program so that the data format of the sixth sub-data is converted into a preset format, and a seventh sub-data is generated and stored as file_temp 2. And then, the fifth sub-data and the seventh sub-data are associated according to the data identification, so that all relevant data are inserted into the third data table.

It should be noted that although the various method steps are shown in FIGS. 2, 3, 5 and 7 in a certain order, this does not mean that the method steps must be performed in the order shown, but rather may be performed in an opposite or parallel order without departing from the spirit and principles of the present disclosure.

The present disclosure also relates to a computer-readable storage medium. The computer-readable storage medium stores computer instructions which, when executed by a processor, implements the data processing method according to any one of the embodiments in FIGS. 2, 3, 5 and 7.

FIG. 9 is a schematic structural diagram showing a data processing device according to an embodiment of the present disclosure. As shown in FIG. 9, the data processing device comprises a memory 91 and a processor 92.

The memory 91 is configured to store instructions, the processor 92 is coupled to the memory 91. The processor 92 is configured to, based on the instructions stored in the memory, execute the data processing method according to any one of the embodiments in FIGS. 2, 3, 5, and 7.

As shown in FIG. 9, the data processing device further comprises a communication interface 93 for information interaction with other devices. Meanwhile, the data processing device further comprises a bus 94. The processor 92, the communication interface 93 and the memory 91 are communicated with each other through the bus 94.

The memory 91 may comprise high-speed RAM memory, and may also comprise non-volatile memory, such as at least one disk memory. The memory 91 may also be a memory array. The storage 91 may also be divided into blocks which may be combined into a virtual volume according to certain rules.

Here, the processor 92 may be a central processing unit (CPU), or may be a general purpose processor, a programmable logic controller (PLC), a digital signal processor (DSP), an application specific integrated circuit (ASIC), a field programmable gate array (FPGA) or other programmable logic device, discrete gate or transistor logic device, discrete hardware components, or any suitable combination thereof that performs the functions described in this disclosure.

Hereto, various embodiments of the present disclosure have been described in detail. Some details well known in the art are not described to avoid obscuring the concept of the present disclosure. According to the above description, those skilled in the art would fully know how to implement the technical solutions disclosed herein. Although some specific embodiments of the present disclosure have been described in detail by way of examples, those skilled in the art should understand that the above examples are only for the purpose of illustration and are not intended to limit the scope of the present disclosure. It should be understood by those skilled in the art that modifications to the above embodiments and equivalently substitution of part of the technical features can be made without departing from the scope and spirit of the present disclosure. The scope of the disclosure is defined by the following claims.

1. A data processing device, comprising:
at least one memory configured to store instructions; and
at least one processor coupled to the at least one memory, and configured to, based on the instructions, perform data processing method comprising:
extracting first data from a first data table in a relational factory database at a first extraction cycle, wherein the first data comprises data updated by the factory during the first extraction cycle, and a duration of the first extraction cycle is greater than 1 minute,
storing the first data into a second data table of a distributed storage system to form second data,
inserting the second data into a third data table of the distributed storage system to form third data after performing data integration on the second data, and
calling the third data in the third data table for data analysis processing at a first analysis cycle, wherein a duration of the first analysis cycle is not smaller than the duration of the first extraction cycle.

2. The data processing device according to claim 1, wherein after the second data is inserted into the third data table to form the third data, the data processing method further comprises:
checking, during a preset time period, the second data inserted into the third data table during a first processing cycle with the first data stored into the second data table during the first processing cycle, such that the second data inserted into the third data table during the first processing cycle is consistent with the data updated in the first data table during the first processing cycle, wherein the duration of the first analysis cycle is greater than a preset threshold during the preset time period.

3. The data processing device according claim 2, wherein the duration of the first extraction cycle ranges from 10 minutes to 1 day.

4. The data processing device according claim 2, wherein checking the second data inserted into the third data table during the first processing cycle with the first data stored into the second data table during the first processing cycle comprises:
performing at least one of deduplication or missing data supplement on the second data inserted into the third data table during the first processing cycle with the first data stored into the second data table during the first processing cycle.

5. The data processing device according claim 2, wherein:
the first data table comprises a first data sub-table and a second data sub-table, and the second data table comprises a third data sub-table and a fourth data sub-table, the first data sub-table comprising first sub-data in the factory database after modification, the second data sub-table comprising second sub-data that is removed during the modification;
extracting the first data from the first data table at the first extraction cycle comprises: extracting the first sub-data from the first data sub-table, and extracting the second sub-data from the second data sub-table at the first extraction cycle;
storing the first data into the second data table comprises: storing the first sub-data into the third data sub-table to form third sub-data, and storing the second sub-data into the fourth data sub-table to form fourth sub-data; and
inserting the second data into the third data table after performing data integration on the second data comprises: inserting the third sub-data into the third data table after performing data integration on the third sub-data.

6. The data processing device according claim 5, wherein the data processing method further comprises:
filtering the third sub-data inserted into the third data table during a second processing cycle with the second sub-data stored into the fourth data sub-table in the second processing period to remove the fourth sub-data inserted into the third data table during the second processing cycle,
wherein a duration of the second processing cycle is greater than the duration of the first processing cycle.

7. The data processing device according claim 2, wherein:
the second data comprises fifth sub-data with a preset data format and sixth sub-data with a compression format; and
inserting the second data into the third data table after performing data integration on the second data comprises:
performing format conversion on the sixth sub-data to obtain seventh sub-data with the preset data format,
associating the fifth sub-data and the seventh sub-data according to a data identifier to obtain fourth data, and
inserting the fourth data into the third data table after performing data integration on the fourth data.

8. The data processing device according claim 7, wherein performing format conversion on the sixth sub-data comprises:
extracting the sixth sub-data from the second data; and
sending the sixth sub-data to a Linux server such that the Linux server performs format conversion on the sixth sub-data to obtain the seventh sub-data with the preset data format.

9. The data processing device according claim 7, wherein the compression format is a BLOB format.

10. A data processing method, comprising:
extracting first data from a first data table in a relational factory database at a first extraction cycle, wherein the first data comprises data updated by the factory during the first extraction cycle, and a duration of the first extraction cycle is greater than 1 minute;
storing the first data into a second data table of a distributed storage system to form second data;
inserting the second data into a third data table of the distributed storage system to form third data after performing data integration on the second data; and
calling the third data in the third data table for data analysis processing at a first analysis cycle, wherein a duration of the first analysis cycle is not smaller than the duration of the first extraction cycle.

11. The data processing method according to claim 10, wherein after inserting the second data into a third data table of the distributed storage system to form third data after performing data integration on the second data, the data processing method further comprises:
checking, during a preset time period, the second data inserted into the third data table during a first processing cycle with the first data stored into the second data table during the first processing cycle, such that the second data inserted into the third data table during the first processing cycle is consistent with the data updated in the first data table during the first processing cycle,
wherein a duration of the first analysis cycle is greater than a preset threshold during the preset time period.

12. The data processing method according to claim 11, wherein the duration of the first extraction cycle ranges from 10 minutes to 1 day.

13. The data processing method according to claim 11, wherein:
the first data table comprises a first data sub-table and a second data sub-table, and the second data table comprises a third data sub-table and a fourth data sub-table, the first data sub-table comprising first sub-data in the factory database after modification, the second data sub-table comprising second sub-data that is removed during the modification;
extracting the first data from the first data table at the first extraction cycle comprises:
extracting the first sub-data from the first data sub-table, and extracting the second sub-data from the second data sub-table at the first extraction cycle;
storing the first data into the second data table comprises:
storing the first sub-data into the third data sub-table to form third sub-data, and storing the second sub-data into the fourth data sub-table to form fourth sub-data;
inserting the second data into the third data table after performing data integration on the second data comprises:
inserting the third sub-data into the third data table after performing data integration on the third sub-data.

14. The data processing method according claim 11, wherein:
the second data comprises fifth sub-data with a preset data format and sixth sub-data with a compression format; and
inserting the second data into the third data table after performing data integration on the second data comprises:
performing format conversion on the sixth sub-data to obtain seventh sub-data with the preset data format,
associating the fifth sub-data and the seventh sub-data according to a data identifier to obtain forth data, and
inserting the fourth data into the third data table after performing data integration on the fourth data.

15. A nonvolatile computer readable storage medium storing computer instructions which, when executed by a processor, perform the data processing method according to claim 10.

16. The data processing method according claim 13, further comprising:
filtering the third sub-data inserted into the third data table during a second processing cycle with the second sub-data stored into the fourth data sub-table in the second processing period to remove the fourth sub-data inserted into the third data table during the second processing cycle, wherein a duration of the second processing cycle is greater than the duration of the first processing cycle.

17. The data processing method according claim 14, wherein performing format conversion on the sixth sub-data comprises:
extracting the sixth sub-data from the second data;
sending the sixth sub-data to a Linux server such that the Linux server performs format conversion on the sixth sub-data to obtain a seventh sub-data with the preset data format;
associating the fifth sub-data and the seventh sub-data according to a data identifier to obtain fourth data; and
inserting the fourth data into the third data table after performing data integration on the fourth data.

18. The nonvolatile computer readable storage medium according to claim 15, wherein after inserting the second data into a third data table of the distributed storage system to form third data after performing data integration on the second data, the data processing method further comprises:
checking, during a preset time period, the second data inserted into the third data table during a first processing cycle with the first data stored into the second data table during the first processing cycle, such that the data inserted into the third data table during the first processing cycle is consistent with the data updated in the first data table during the first processing cycle,
wherein a duration of the first analysis cycle is greater than a preset threshold during the preset time period.

19. The nonvolatile computer readable storage medium according to claim 15, wherein:
the first data table comprises a first data sub-table and a second data sub-table, and the second data table comprises a third data sub-table and a fourth data sub-table, the first data sub-table comprising first sub-data in the factory database after modification, the second data sub-table comprising second sub-data that is removed during the modification;
extracting the first data from the first data table at the first extraction cycle comprises:
extracting the first sub-data from the first data sub-table, and extracting the second sub-data from the second data sub-table at the first extraction cycle;
storing the first data into the second data table comprises:
storing the first sub-data into the third data sub-table to form third sub-data, and storing the second sub-data into the fourth data sub-table to form fourth sub-data;
inserting the second data into the third data table after performing data integration on the second data comprises:
inserting the third sub-data into the third data table after performing data integration on the third sub-data.

20. The nonvolatile computer readable storage medium according to claim 19, wherein:
the second data comprises fifth sub-data with a preset data format and sixth sub-data with a compression format; and
inserting the second data into the third data table after performing data integration on the second data comprises:
performing format conversion on the sixth sub-data to obtain seventh sub-data with the preset data format,
associating the fifth sub-data and the seventh sub-data according to a data identifier to obtain fourth data, and
inserting the fourth data into the third data table after performing data integration on the fourth data.
